(19) United States
(12) Patent Application Publication
(10) Pub. No.: US 2005/0209303 A1
(43) Pub. Date: Sep. 22, 2005

(54) Method of treatment for high neurotransmitter pattern

(76) Inventor: Gottfried Kellermann, Osceola, WI (US)

Correspondence Address: DOUGHERTY, CLEMENTS, HOFER, BERNARD & WALKER, 1901 ROXBOROUGH ROAD, SUITE 300, CHARLOTTE, NC, 28211, US

(21) Appl. No.: 10/806,033

(22) Filed: Mar. 22, 2004

(51) Int. Cl. A61K 31/405; A61K 31/198; A61K 31/185

(52) U.S. Cl. 514/419; 514/553; 514/567; 514/566; 514/563

(57) ABSTRACT

A person suffering from a high neurotransmitter pattern, as is characteristic with a patient diagnosed with ADD or ADHD, is treated by administering to the person an effective amount of: 5-hydroxytryptophan, L-theanine, and a neurotransmitter modulator selected from the group consisting of L-taurine and L-glutamine

### REFERENCE TO RELATED APPLICATIONS

This application is related to Provisional Application Ser. No. 60/319,965 filed Feb. 21, 2003 for “Nutritional Composition and Method for Treating ADD or ADHD.”

### BACKGROUND OF THE INVENTION

The present invention relates to a method of treatment for persons having a pattern of high neurotransmitter levels. Such persons often exhibit symptoms associated with Attention Deficit Disorder (ADD) or Attention Deficit Hyperactivity Disorder (ADHD). The method of treatment builds a strong inhibitory neurotransmitter system in the patient by administering inhibitory neurotransmitters and neurotransmitter modulators to the patient.

Attention Deficit Disorder, hereinafter referred to as “ADD,” and Attention Deficit Hyperactivity Disorder, hereinafter referred to as “ADHD,” are conditions which are diagnosed when specific behaviors, such as inattentiveness, hyperactivity, or impulsivity have a significant impairment on social, academic, or occupational function and development. ADD and ADHD are nearly identical, differing only by the addition of hyperactivity to ADHD. The conditions are more commonly found in children than in adults.

Diagnosis of ADD or ADHD is somewhat subjective and there is much debate among clinical practitioners concerning these conditions and their diagnosis. As with most conditions, there are varying degrees of severity of ADD or ADHD. However, it has been found that there is a recurring neurotransmitter pattern displayed by children with ADD or ADHD symptoms. More specifically, it has been found that the majority of ADD or ADHD patients will have higher than normal levels of the following neurotransmitters:

| Transmitter | Test Result Level |
|---|---|
| epinephrine | high |
| norepinephrine | high |
| dopamine | very high |
| serotonin | high or low |
| GABA | high |
| PEA | high |

The typical ADD/ADHD patient generally displays high levels of each of the above neurotransmitters relative to established reference ranges for timed morning urine specimens. A notably high transmitter level is the elevated dopamine level.

Investigations have shown that high dopaminergic stimulation of the prefrontal cortex significantly disrupts working memory. Stress also increases dopaminergic activity in the prefrontal cortex. This may explain why low levels of stress can enhance memory, but elevated levels of dopamine can disrupt performance. Since memory is a significant factor in the learning process, therapeutic approaches that address dopamine balance are vitally important in the patient's treatment.

The above test results refer to the result of testing for levels of neurotransmitters in patient's urine. High neurotransmitter levels in urine are interpreted as “high activity” within the body as the body is apparently using and excreting excessive amounts of neurotransmitters in an unregulated fashion. High neurotransmitter activity is believed to result in the clinical presentation of ADD or ADHD symptoms: lack of focus, inattentiveness, memory problems, hyperactivity, etc.

While the exact cause of high neurotransmitter activity is hard to pinpoint, it is generally thought to be the result of a number of factors including diet, genetics, stress, and other environmental influences. Whatever its cause, if left untreated, the excessive excretion of transmitters eventually leads to depletion of neurotransmitter reserves. The body cannot maintain the heightened level of excretion for an extended period of time without health-related consequences.

Accordingly, there is a need for a treatment method to treat those having a pattern of high neurotransmitter levels as evidenced by their excessive excretion of neurotransmitters. In accordance with the present invention, an effective method is provided to persons having a high neurotransmitter pattern, such as ADD and ADHD patients, with specific amino acid precursors and modulators that decrease the excretion (activity) of the neurotransmitters and promote the regeneration of neurotransmitter stores. Symptom resolution often results from the present treatment method which involves the administration of 5-hydroxytryptophan, L-theanine, and one or more compounds selected from the group consisting of tyrosine, N-acetyl-L-tyrosine, and phenyl alanine. Preferably, the method also includes the administration of neurotransmitter modulators L-taurine and L-glutamine. Further understanding of the present invention will be had from the following specification and claims. All parts and percentages herein are by weight unless otherwise indicated.

### BRIEF SUMMARY OF THE INVENTION

In accordance with the method of the present invention, a person suffering from a high neurotransmitter pattern is treated by administering to the person therapeutically effective amounts of 5-hydroxytryptophan, L-theanine, and one or more compounds selected from the group consisting of tyrosine, N-acetyl-L-tyrosine, and phenyl alanine. Preferably, the method also includes the administration of therapeutically effective amounts of neurotransmitter modulators L-taurine and L-glutamine. Preferably the treatment method comprises three phases: phase 1 of from 2 to 4 weeks of treatment, phase 2 of from 1 to 3 months of treatment and phase 3, a maintenance phase, of from several months to several years. In the first phase, 5-HTP and L-theanine are administered. In the second phase, 5-HTP, L-theanine and at least one compound selected from the group consisting of tyrosine, N-acetyl-L-tyrosine, and phenyl alanine are administered. Preferably, L-taurine and/or L-glutamine are also administered in the second phase. In the third phase, 5-HTP, L-theanine, and at least one compound selected from the group consisting of tyrosine, N-acetyl-L-tyrosine, and phenyl alanine, preferably also L-taurine and/or L-glutamine are administered, but the amount of 5-HTP administered is reduced.

### DETAILED DESCRIPTION OF THE INVENTION

The present invention provides a method for treatment for a person having a high neurotransmitter pattern, such as is typical of a patient with ADD or ADHD. Thus, a patient having a high neurotransmitter pattern is treated by administering to the person therapeutically effective amounts of 5-hydroxytryptophan, L-theanine, and one or more compounds selected from the group consisting of tyrosine, N-acetyl-L-tyrosine, and phenyl alanine. Preferably, the method also includes the administration of therapeutically effective amounts of neurotransmitter modulators L-taurine and L-glutamine. Preferably the treatment method comprises three phases: phase 1 of from 2 to 4 weeks of treatment, phase 2 of from 1 to 3 months of treatment and phase 3, a maintenance phase, of from several months to several years. In the first phase, 5-HTP and L-theanine are administered. In the second phase, 5-HTP, L-theanine and at least one compound selected from the group consisting of tyrosine, N-acetyl-L-tyrosine, and phenyl alanine are administered. Preferably, L-taurine and/or L-glutamine are also administered in the second phase. In the third phase, 5-HTP, L-theanine, and at least one compound selected from the group consisting of tyrosine, N-acetyl-L-tyrosine, and phenyl alanine, preferably also L-taurine and/or L-glutamine are administered, but the amount of 5-HTP administered is reduced. In each case, therapeutically effective amounts are administered.

It has been found that the administratinon of 5-hydroxytryptophan causes regeneration of serotonin stores and thus imparts the calming effect of serotonin to the patient. L-theanine decreases neurotransmitter activity, particularly dopamine activity, but also serotonin, epinephrine, norepinephrine, and PEA (phenylethylamine) activity. L-taurine has a positive GABA receptor modulation effect. L-glutamine is a precursor to GABA.

The main focus of the approach of the method of this invention is to build a strong inhibitory neurotransmitter system. The inhibitory neurotransmitter system includes the neurotransmitters serotonin and GABA, and is responsible for regulating excitatory transmitters such as PEA, epinephrine, norepinephrine, and dopamine. A properly functioning inhibitory system filters out unnecessary signals sent by the excitatory system. When the inhibitory system is dysfunctional, the excitatory system fires excessively, leading to high neurotransmitter levels and to the symptoms of ADD/ADHD described above.

It will be appreciated by those skilled in the art that each of these compounds administered in accordance with the present invention is known and commercially available. Furthermore, it will be appreciated that the compounds can be administered to the person or patient in any conventional manner such as oral administration in pill form or the like. L-theanine can be particularly well administered in the form as a liposomal spray as is disclosed in my copending application Ser. No. 10790619 filed Mar. 1, 2004, for LIPOSOMAL COMPOSITON COMPRISING L-THEANINE. The compositions can be administered together or sequentially, with the aforementioned phase protocol being preferred.

It will also be appreciated that a number of other components regulate the balance of excitatory and inhibitory transmitters and that selected such other modulators of this balance may be included in the present composition. These include other amino acids as well as vitamin and mineral components that may address neurotransmitter synthesis, release, or function such as magnesium, calcium, chromium, selenium, folic acid, riboflavin, pantothenic acid, vitamin B6, B12, and C.

The following examples are intended to illustrate, but not limit, the present invention.

### EXAMPLE 1

Capsules are made up of the following ingredients:

Capsules A

| | |
|---|---|
| 5-Hydroxytryptophan | 100 mg |
| Vitamin C | 300 mg |
| Calcium (calcium citrate) | 73.5 mg |
| Folate | 133 mcg |
| Vitamine B6 (Pyridoxine HCl & P-5-P) | 25 mg |

Capsules B

| GABAMax 90 ct | /cap |
|---|---|
| N-acetyl-L-Tyrosine | 50 mg |
| 5-Hydroxytryptophan | 50 mg |
| Theanine | 75 mg |
| L-Glutamine | 350 mg |
| Taurine | 250 mg |
| Vitamine B6 (Pyridoxine HCL & P-5-P) | 12.5 mg |
| Folic Acid | 67 mg |
| Vitamin C | 21 mg |
| Magnesium Glycinate | 50 mg |

A liposomal spray composition was made up comprising L-Theanine in a liposomal spray bottle providing 50 mg of L-theanine per spray.

The above compositions are administered to a patient having ADD symptoms as follows:

| | | | |
|---|---|---|---|
| for three weeks: | Capsules A | 2 caps BID | AM, HS |
| | L-theanine | 2 sprays | 3-5 times daily |
| for two months: | Capsules A | 2 caps BID | AM, HS |
| | Capsules B | 1 cap BID | AM, PM |
| | L-theanine | 2 sprays | as needed |
| for one year: | Capsules B | 1 cap BID | AM, PM |
| | L-theanine | 2 sprays | as needed |

A substantial reduction of ADD symptoms is observed in the patient.

### EXAMPLE 2

The regimen of Example 1 is followed except that Capsule B is replaced by a Capsule which differs from Capsule B by the substitution of N-acetyl-L-Tyrosine with Phenylalanine.

Of course, it will b appreciated by those skilled in the art that the present invention described above is subject to modification and variation and that it is intended that this invention be limited only by the scope of the following claims.

1. A method of treatment of a person having a high neurotransmitter level pattern, comprising the step of administering to the person therapeutically effective amounts of 5-hydroxytryptophan, L-theanine, and one or more compounds selected from the group consisting of tyrosine, N-acetyl-L-tyrosine, and phenyl alanine.

2. The method of claim 1 including administrating therapeutically effective amounts of neurotransmitter modulators L-taurine and L-glutamine.

3. The method of claim 1 wherein said administrating is carried out in three timed phases, a first phase of from 2 to 4 weeks of treatment, a second phase of from 1 to 3 months of treatment and a third phase of from several months to several years, with different compositions being administered in each of said phases.

4. The method of claim 3, wherein in said first phase, 5-HTP and L-theanine are administered, but a compound from the group consisting of tyrosine, N-acetyl-L-tyrosine, and phenyl alanine is not administered.

5. The method of claim 4, wherein in said second phase, 5-HTP, L-theanine and at least one compound selected from the group consisting of tyrosine, N-acetyl-L-tyrosine, and phenyl alanine are administered.

6. The method of claim 5, wherein, in said second phase, at least one compound selected from the group consisting of taurine and/or L-glutamine are also administered.

7. The method of claim 5 wherein in the third phase, 5-HTP, L-theanine, and at least one compound selected from the group consisting of tyrosine, N-acetyl-L-tyrosine, and phenyl alanine are administered, but the amount of 5-HTP administered is less than that administered during said first phase.

8. The method of claim 5 wherein at least one compound selected from the group consisting of taurine and/or L-glutamine is administered.

9. The method of claim 7 wherein at least one compound selected from the group consisting of taurine and/or L-glutamine is administered.
